(19) United States
(12) Patent Application Publication
(10) Pub. No.: US 2024/0247869 A1
(43) Pub. Date: Jul. 25, 2024

(54) ENDOSCOPE DRYING AND DRYING VERIFICATION DEVICE

(71) Applicant: American Sterilizer Company, Mentor, OH (US)

(72) Inventors: Amy K. Williams, Hendersonville, TN (US); Robert W. Williams, Hendersonville, TN (US)

(21) Appl. No.: 18/421,317

(22) Filed: Jan. 24, 2024

Related U.S. Application Data
(60) Provisional application No. 63/481,275, filed on Jan. 24, 2023.

(51) Int. Cl. F26B 5/16; G01N 21/81

(52) U.S. Cl. CPC F26B 5/16; G01N 21/81

(57) ABSTRACT

Various embodiments disclosed relate to an endoscope drying device that also provides an indication of lumen drying verification. The present disclosure includes a device having an elongated and flexible base having a proximal portion and a distal portion and a hydrochromic absorbent unit extending from the distal portion. The hydrochromic absorbent unit can be configured to visually indicate when fluid is present. The hydrochromic absorbent unit can be sized and shaped for insertion into an endoscope channel. When inserted into the endoscope channel, the hydrochromic absorbent unit can expand to fill the endoscope channel and absorb any moisture therein.

### PRIORITY CLAIM

This application claims priority and benefit of U.S. Provisional Application with Ser. No. 63/481,275, filed Jan. 24, 2023, entitled ENDOSCOPE DRYING AND DRYING VERIFICATION DEVICE, which is herein incorporated by reference in its entirety.

### BACKGROUND

Endoscopic devices are used for numerous medical procedures. For example, endoscopes can be used for removal of lesions, polyps, or other targeted tissue. In such procedures, and in other endoscopic procedures, the endoscope used may be contaminated, both internally and externally. Such an endoscope is typically reprocessed to clean and perform high-level disinfection of the endoscope. Devices that decontaminate the endoscope can help to fully clean the endoscope for future procedures, as well as lengthen the life of the endoscope.

After an endoscope is used on a patient, a number of steps are done to reprocess the endoscope before the contaminated endoscope can be reused. Reprocessing steps can include cleaning and disinfecting steps, such as with liquid disinfectant solution. Automated Endoscope Reprocessors (AERs) can be used in healthcare settings to reprocess lumen-containing medical devices such as endoscopes, including robotic endoscopic instruments, flexible and rigid type endoscopes, such as duodenoscopes, and endoscope accessories. AERs are designed to kill microorganisms in or on reusable endoscopes by exposing their outside surfaces and interior channels to chemical solutions.

Long, narrow channels of endoscopes can be particularly challenging to reprocess. Moreover, when such channels and lumens are reprocessed, such as with liquid disinfectant solution, it can be challenging to determine when the lumen has been properly dried. If not properly dried, microbes, disinfectant liquid, water, or other residual fluid items can remain in the lumen, causing potential problems for disinfection and function.

### SUMMARY OF THE DISCLOSURE

The present disclosure provides an endoscope channel drying device and a method of use thereof. The device can allow for both drying and confirmation of drying of any residual liquid which remains in an endoscope channel after reprocessing. The device can include a catheter, with an absorbent material, such as a sponge-like material, on a distal end. The sponge-like material can be pliable and moved through a channel of an endoscope for which drying is desired. The sponge-like material can be traversed (e.g., pushed or pulled) through the channel of the endoscope. The sponge-like material can be a hydrochromic material that color changes when in contract with fluid. Thus, once the catheter is pushed through, and the sponge-like material has run through the endoscope channel, a user will be able to visually see color change. Correspondingly, the user can determine if any moisture was left in the channel. The excess moisture can be removed by the sponge-like material.

In an example, a device can include: an elongated and flexible base having a proximal portion and a distal portion; and a hydrochromic absorbent unit extending from the distal portion, the hydrochromic absorbent unit configured to visually indicate when fluid is present, the hydrochromic absorbent unit sized and shaped for insertion into an endoscope channel, wherein when inserted into the endoscope channel, the hydrochromic absorbent unit expands to fill the endoscope channel and absorb any moisture therein.

In an example, a method of drying an endoscope channel can include traversing a device including an elongated base and a pliable hydrochromic material into the endoscope channel; running the device through the endoscope channel to soak up any excess moisture; and observing color of the hydrochromic material upon exit from the endoscope channel to determine whether excess moisture was present in the endoscope channel.

In an example, a kit can include: an elongated and flexible body for insertion into a lumen with a drying portion attached to the elongated and flexible body, the drying portion comprising a material configured to change color when in contact with moisture, wherein the drying portion is configured to expand within the lumen when inserted, such that the drying portion contacts walls of the lumen and absorbs moisture therein.

### BRIEF DESCRIPTION OF THE DRAWINGS

In the drawings, which are not necessarily drawn to scale, like numerals may describe similar components in different views Like numerals having different letter suffixes may represent different instances of similar components. The drawings illustrate generally, by way of example, but not by way of limitation, various embodiments discussed in the present document.

FIG. 1 illustrates a view of a device for endoscope drying and indication in an example.

FIG. 2 illustrates a method of using a drying device for endoscope drying and indication in an example.

FIG. 3 illustrates a method of endoscope processing in an example.

### DETAILED DESCRIPTION

The present disclosure describes, among other things, an endoscope drying device that can be used for both drying and drying verification of lumens. The device can help confirm drying of an endoscope channel via a hydrochromic material that allows color change to occur when then material encounters any moisture. The color change can indicate there is moisture and the drying process should continue. The absence of color change can indicate moisture is not present. This device can be used on flexible or non-flexible endoscopes, such as colonoscopes.

The devices and methods herein can allow for immediate drying verification, reducing delays in the drying process. The devices can be disposable, single use device for one pass through a single endoscope channel. The device does not require any other capital or consumable products to perform a drying verification test. This can help reduce drying time, increase productivity, and promote staff confidence by removing the potential for misinterpretation of findings from visual inspection for moisture.

Cleaning and drying verification can be challenging for a large number of medical devices, and particularly for medical devices with lumens. Checking both the external surfaces of a device, and internal surfaces such as lumens, for residual contaminants or fluids can be challenging to quickly and accurately accomplish. Reaching the interior spaces of lumens can be difficult, moreover, ensuring the entire interior surface of the lumen is dry and free of contaminants or fluids is prohibitive due to the small diameters and long length of such lumens in endoscopes.

The proposed device herein allows for simultaneous drying and confirmation of drying of an endoscope lumen. The device can include a sponge-like material that conforms to the interior contours of an endoscope lumen, such as to allow drying and verification of crevices and contours therein. The device can be pulled through the length of an endoscope lumen to dry along the lumen itself.

Simultaneously, the sponge-like material can be made of a water-sensitive material, such as a hydrochromic material which color-changes with exposure to water. This can allow for easy visual inspection upon drying of the endoscope lumen to determine whether or not drying has been completed.

The device is a unidirectional device that can be pushed thru the suction and biopsy channels of an endoscope, starting at proximal end and removed from the distal end of the endoscope. Dry and clean exam gloves can be used during this process. After removal, the sponge can be examined for any color change which will occur if the sponge comes in contact with moisture while being passed through the endoscope. If a color change is noted, a new sponge can be obtained and the process repeated until no color change is detected. This can help verify that all channels of an endoscope are completely dry prior to placing the endoscope in a storage cabinet or returning them for patient use.

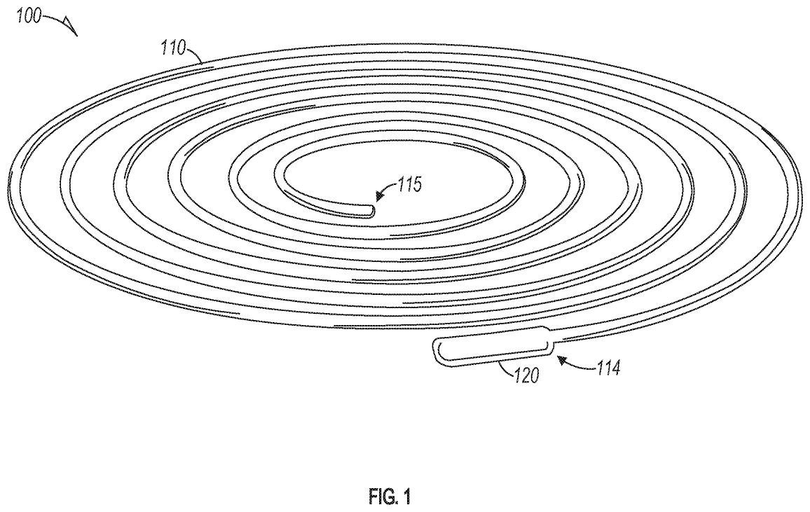

FIG. 1 illustrates an example device 100. The device 100 can include an elongated flexible base 110 with a proximal portion 112 and a distal portion 114, in addition to a hydrochromic absorbent unit 120. The hydrochromic absorbent unit 120 can extend from the distal portion 114. The device 100 can be sized and shaped for insertion into an endoscope lumen, such as a suction channel or a biopsy channel.

The elongated flexible base 110 can be, for example, a polymer material stiff enough for insertion and threading through the length of an endoscope channel, such as from a proximal end of the endoscope towards the distal end of the endoscope. The elongated flexible base 110 can be both sturdy enough to push through a lumen of the endoscope and flexible enough to navigate contours of the endoscope lumen. In an example, the elongated flexible base 110 can be a polymer catheter. In an example, the elongated flexible base 110 can be a twisted polymer cable. In an example, the elongated flexible base 110 can be about 200 cm to about 250 5 cm in length.

The hydrochromic absorbent unit 120 can extend from the distal portion 114 of the elongated flexible base 110. The hydrochromic absorbent unit 120 can be configured to visually indicate when fluid is present within a lumen once applied. The hydrochromic absorbent unit 120 can be sized and shaped for insertion into an endoscope channel. When inserted into the endoscope channel, the hydrochromic absorbent unit 120 can expand to fill the endoscope channel and absorb any moisture therein.

In an example, the hydrochromic absorbent unit 120 can have a diameter of about 1.0 to about 1.2 mm. In an example, the hydrochromic absorbent unit 120 can have a diameter of about 1.5 to about 2.5 mm. In an example, the hydrochromic absorbent unit 120 can have a diameter of about 2.6 to about 4.8 mm. Variations on the size of the hydrochromic absorbent unit 120 can be used with different sized and shaped endoscope lumens.

The hydrochromic absorbent unit 120 can be made of squishable, resilient material that can fit into an endoscope lumen and expand to fill the lumen space. The hydrochromic absorbent unit 120 can be made of a color-changing hydrochromic material that changes color to visually indicate when it has come into contact with moisture. In an example, the hydrochromic absorbent unit 120 can be a hydrochromic fabric. In an example, the hydrochromic absorbent unit 120 can be a hydrochromic ink or hydrochromic dye infused material. In an example, the hydrochromic absorbent unit 120 can be a sponge material.

The hydrochromic absorbent unit 120 can circumferentially surrounds the distal portion 114 of the elongated flexible base 110. The hydrochromic absorbent unit 120 can surround and extends outward from the distal portion 114 of the elongated flexible base 110.

In an example, the device can be made in a variety of sizes appropriate for different endoscopes. For example, the hydrochromic material can be, for example, a small 1.0-1.2 mm Gold colored sponge, a medium 1.5-2.5 mm Blue colored sponge, or a large 2.6-4.8 mm Green colored sponge. In an example, these sponges can be 12 mm long.

In some examples, the device can be a kit. The kit can include individual components, each of which is sterilized. In such cases, the kit can include an elongated and flexible body for insertion into a lumen and a drying portion attachable to the elongated and flexible body. The drying portion can include a material configured to change color when in contact with moisture. The drying portion can be configured to expand within the lumen when inserted, such that the drying portion contacts walls of the lumen and absorbs moisture therein. The color-changing material can be a hydrochromic pigment hosted in a substrate, the hydrochromic pigment configured to change color when in contact with moisture.

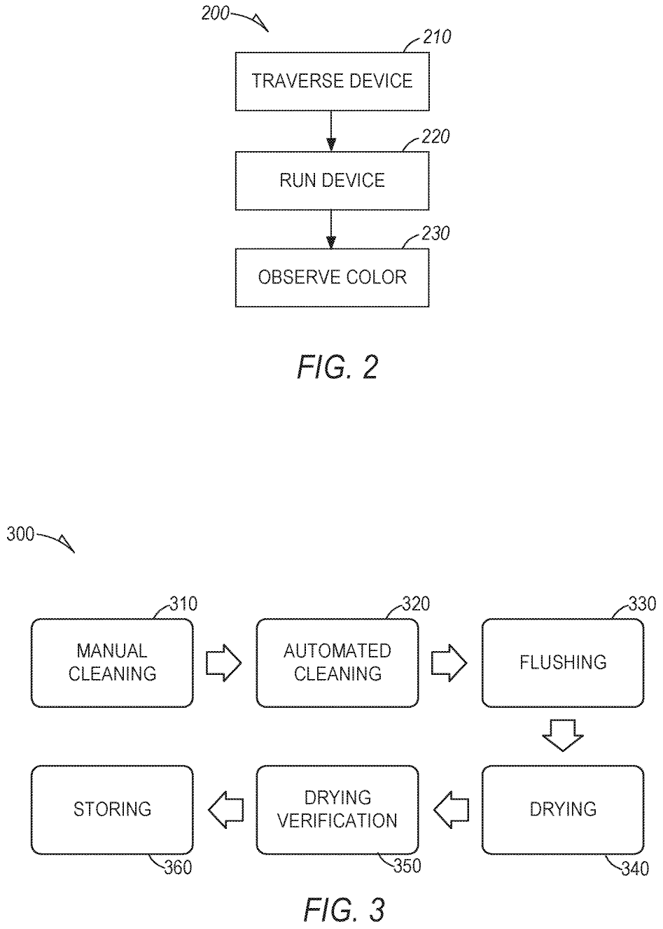

FIG. 2 illustrates a method 200 of using a drying device for endoscope drying and indication in an example. The method 200 can include blocks 210, 220, and 230.

At block 210, the method can include traversing a device including an elongated base and a pliable hydrochromic material into the endoscope channel. Traversing the device can inserting the device at a proximal end of the endoscope channel and pushing the device therethrough. The method can optionally include selecting the device based on a size of the endoscope channel.

At block 220, the method can include running the device through the endoscope channel to soak up any excess moisture. Running the device can include pulling the device through a distal end of the endoscope channel.

At block 230, the method can include observing color of the hydrochromic material upon exit from the endoscope channel to determine whether excess moisture was present in the endoscope channel. Observing color of the hydrochromic material can include comparing color of the hydrochromic material upon removal of the device from the channel to color of the hydrochromic material upon insertion in the channel. The method can optionally include repeating the method if moisture was detected in the endoscope channel.

The method 200 can be performed at room temperature, in normal humidity environments. The device or device kits can be, for example, single-use devices that are packaged and sterilized individually and used after opening.

FIG. 3 illustrates a method 300 of endoscope reprocessing in an example. The method can include blocks 310 to 360.

First, at block 310 the endoscope is manually cleaned. For example, the external surfaces of the endoscope can be wiped, and the interior channels of the endoscope can be cleaned with conventional mechanical tools.

Next, at block 320, the endoscope is reprocessed with a disinfecting solution in an automated machine. An automated endoscope reprocessor (AER) can be used here. Here, the endoscope channel(s) can be hooked up to the AER. The AER can be programmed to run disinfectant solutions through the various channels of the endoscope. Next, at block 330, the disinfection solution is flushed. For example, the automated endoscope reprocessor can push rinse water through the various channels of the endoscope and flush out remaining disinfection solution. In some cases, cleaning and disinfecting solutions can be used. In some cases, air purge can be used.

Next, at block 340, the endoscope is purged with air. In this step, the various channels of the endoscope can be purged, such as with sterile air therethrough. In some cases, the endoscope can be disconnected from the automated endoscope reprocessor at this step. This can be done, for example, through other drying devices.

By contrast, the drying device discussed herein, which can be used for both drying and drying verification, can be used in addition to other endoscope reprocessing and air purging, as a supplement. In this case, the device with the hydrochromic material can be pushed through each channel of the endoscope to help remove excess moisture.

At block 350, the drying device discussed herein can be used, as a supplement to and after endoscope reprocessing. Once done with the supplemental drying of the device discussed herein, endoscope drying is verified. This can be done, for example, by visual inspection of the hydrochromic material, to determine whether any color change has occurred, and whether additional drying may be desired. Finally, at block 360, the endoscope is stored.

### VARIOUS NOTES & EXAMPLES

Example 1 is a device comprising: an elongated and flexible base having a proximal portion and a distal portion; and a hydrochromic absorbent unit extending from the distal portion, the hydrochromic absorbent unit configured to visually indicate when fluid is present, the hydrochromic absorbent unit sized and shaped for insertion into an endoscope channel, wherein when inserted into the endoscope channel, the hydrochromic absorbent unit expands to fill the endoscope channel and absorb any moisture therein.

In Example 2, the subject matter of Example 1 optionally includes wherein the hydrochromic absorbent unit is about 1.0 mm to about 1.2 mm in diameter.

In Example 3, the subject matter of any one or more of Examples 1-2 optionally includes wherein the hydrochromic absorbent unit is about 1.5 mm to about 2.5 mm in diameter.

In Example 4, the subject matter of any one or more of Examples 1-3 optionally includes wherein the hydrochromic absorbent unit is about 2.6 mm to about 4.8 mm in diameter.

In Example 5, the subject matter of any one or more of Examples 1-4 optionally includes wherein the elongated and flexible base is about 200 cm to about 250 cm in length.

In Example 6, the subject matter of any one or more of Examples 1-5 optionally include wherein the hydrochromic absorbent unit comprises a hydrochromic fabric.

In Example 7, the subject matter of any one or more of Examples 1-6 optionally include wherein the hydrochromic absorbent unit comprises a hydrochromic ink or hydrochromic dye infused material.

In Example 8, the subject matter of any one or more of Examples 1-7 optionally include wherein the hydrochromic absorbent unit comprises a sponge.

In Example 9, the subject matter of any one or more of Examples 1-8 optionally include wherein the elongated and flexible base comprises a polymer catheter.

In Example 10, the subject matter of any one or more of Examples 1-9 optionally include wherein the elongated and flexible base comprises a twisted polymer cable.

In Example 11, the subject matter of any one or more of Examples 1-10 optionally include wherein the hydrochromic absorbent unit circumferentially surrounds the distal portion of the elongated and flexible base.

In Example 12, the subject matter of any one or more of Examples 1-11 optionally include wherein the hydrochromic absorbent unit surrounds and extends outward from the distal portion of the elongated flexible base.

Example 13 is a method of drying an endoscope channel, the method comprising: traversing a device including an elongated base and a pliable hydrochromic material into the endoscope channel; running the device through the endoscope channel to soak up any excess moisture; and observing color of the hydrochromic material upon exit from the endoscope channel to determine whether excess moisture was present in the endoscope channel.

In Example 14, the subject matter of any one or more of Examples 11-13 optionally include wherein pushing the device comprises inserting the device at a proximal end of the endoscope channel.

In Example 15, the subject matter of any one or more of Examples 11-14 optionally include wherein running the device comprises pulling the device through a distal end of the endoscope channel.

In Example 16, the subject matter of any one or more of Examples 11-15 optionally include wherein observing color of the hydrochromic material comprises comparing color of the hydrochromic material upon removal of the device from the channel to color of the hydrochromic material upon insertion in the channel.

In Example 17, the subject matter of any one or more of Examples 11-16 optionally include selecting the device based on a size of the endoscope channel.

In Example 18, the subject matter of any one or more of Examples 11-17 optionally include repeating the method if moisture was detected in the endoscope channel.

Example 19 is a kit comprising: an elongated and flexible body for insertion into a lumen with a drying a drying portion attached to the elongated and flexible body, the drying portion comprising a material configured to change color when in contact with moisture, wherein the drying portion is configured to expand within the lumen when inserted, such that the drying portion contacts walls of the lumen and absorbs moisture therein.

In Example 20 the subject matter of claim 19 optionally includes wherein the color-changing material comprises a hydrochromic pigment hosted in a substrate, the hydrochromic pigment configured to change color when in contact with moisture.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part, including in some settings with the use of automation, robotics, or other controlled electronic systems. The use of such methods in computer-implemented or electronic-systems can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device comprising:
an elongated and flexible base having a proximal portion and a distal portion; and
a hydrochromic absorbent unit extending from the distal portion, the hydrochromic absorbent unit configured to visually indicate when moisture is present, the hydrochromic absorbent unit sized and shaped for insertion into an endoscope channel, wherein when inserted into the endoscope channel, the hydrochromic absorbent unit fills the endoscope channel and absorb any moisture therein.

2. The device of claim 1, wherein the hydrochromic absorbent unit is about 1.0 to about 1.2 mm in diameter.

3. The device of claim 1, wherein the hydrochromic absorbent unit is about 1.5 to about 2.5 mm in diameter.

4. The device of claim 1, wherein the hydrochromic absorbent unit is about 2.6 to about 4.8 mm in diameter.

5. The device of claim 1, wherein the elongated and flexible base is about 200 cm to about 250 cm in length.

6. The device of claim 1, wherein the hydrochromic absorbent unit comprises a hydrochromic fabric.

7. The device of claim 1, wherein the hydrochromic absorbent unit comprises a hydrochromic ink or hydrochromic dye infused material.

8. The device of claim 1, wherein the hydrochromic absorbent unit comprises a sponge.

9. The device of claim 1, wherein the elongated and flexible base comprises a polymer catheter.

10. The device of claim 1, wherein the elongated and flexible base comprises a twisted polymer cable.

11. The device of claim 1, wherein the hydrochromic absorbent unit circumferentially surrounds the distal portion of the elongated and flexible base.

12. The device of claim 1, wherein the hydrochromic absorbent unit surrounds and extends outward from the distal portion of the elongated and flexible base.

13. A method of drying an endoscope channel, the method comprising:
traversing a device including an elongated base and a pliable hydrochromic material into the endoscope channel;
running the device through the endoscope channel to soak up any excess moisture; and
observing color of the hydrochromic material upon exit from the endoscope channel to determine whether excess moisture was present in the endoscope channel.

14. The method of claim 13, wherein pushing the device comprises inserting the device at a proximal end of the endoscope channel.

15. The method of claim 13, wherein running the device comprises pulling the device through a distal end of the endoscope channel.

16. The method of claim 13, wherein observing color of the hydrochromic material comprises comparing color of the hydrochromic material upon removal of the device from the channel to color of the hydrochromic material upon insertion in the endoscope channel.

17. The method of claim 13, further comprising selecting the device based on a size of the endoscope channel.

18. The method of claim 13, further comprising repeating the method if moisture was detected in the endoscope channel.

19. A kit comprising:
an elongated and flexible body for insertion into a lumen of an endoscope; and
a drying portion attachable to the elongated and flexible body, the drying portion comprising a material configured to change color when in contact with moisture,
wherein the drying portion is configured to expand within the lumen when inserted, such that the drying portion contacts walls of the lumen and absorbs moisture therein.

20. The kit of claim 19, wherein the color-changing material comprises a hydrochromic pigment hosted in a substrate, the hydrochromic pigment configured to change color when in contact with moisture.
